SEQUENCE LISTING (11 sequences)

SEQ ID NO: 1 - DNA, 101 bp, Solanum lycopersicum
  source 1..101: mol_type = genomic DNA
  variation 51: note = Resistant = C; Susceptible = T
tgcgctaggatatttgaatgttatttatccaacaaaaatgttacacactgycaattaaaaatcaaattctaatactcttgctggctcttgaaagaaataaa

SEQ ID NO: 2 - DNA, 301 bp, Solanum lycopersicum
  source 1..301: mol_type = genomic DNA
  variation 151: note = Resistant = C; Susceptible = A
caacggtatngctctccatccaaggagcatcattataaacaagattaccagcactngtaagaactccagaagaatcaggaagtaataaaggagtaccagaanactcaaatattaagctatccatgttgcaatctgcaattgcttctagcamgtgattgacaaaactcagttgatcggcagaaagggggnatcctttcacatcattttgcaacctttgtaaaacatggaagtaatcaaaaacatcaaaactgagtctaacgcccaactccacaagcaaatctctgaaatccgttaactcaga

SEQ ID NO: 3 - DNA, 301 bp, Solanum lycopersicum
  source 1..301: mol_type = genomic DNA
  variation 151: note = Resistant = G; Susceptible = A
gaattgttcagtaagtgtgcttgattcctggcagattaaattagtctgatagtgcttaggtgatccaatgccatattctcaagttaacatttgttttggtcgtggcttattnttgtaaaattaattgggttgatcccaataggaaatattratgattgattgtaggcatccttctttcacagatgttattggctttgcctatgtattcactatattactattgtatgtgttctntagtctagccaattgtgtggttgtaggttaatgttagctcctttttgctctgggcttcttactcaac

SEQ ID NO: 4 - DNA, 121 bp, Solanum lycopersicum
  source 1..121: mol_type = genomic DNA
  variation 61: note = Resistant = C; Susceptible = A
gtgaaatccattganatccctcctcctcggcctaaaagaaagccgatgcacccttatccamgaaaaatggcaactccacttaaaagtggaaccctggcttcagagaaattgaacagatctg

SEQ ID NO: 5 - DNA, 101 bp, Solanum lycopersicum
  source 1..101: mol_type = genomic DNA
  variation 51: note = Resistant = T; Susceptible = C
taatgtctagacagagatagtatgacagaaattcgttctagcttcagattyttagttatctttcaaaattatgaaacattacattaagggataaagccatc

SEQ ID NO: 6 - DNA, 121 bp, Solanum lycopersicum
  source 1..121: mol_type = genomic DNA
  variation 61: note = Resistant = C; Susceptible = T
cattcttaatagtggatgctatattcaaatcttaatataatcggtgttttattaaccttayccttgagttaaaaattgataaggtcttcttttcaagagttacttaaatcggattctggag

SEQ ID NO: 7 - DNA, 301 bp, Solanum lycopersicum
  source 1..301: mol_type = genomic DNA
  variation 151: note = Resistant = C; Susceptible = G
aatttcatctcaccaccaaatatgattgttatccggtcatttgatgtaaataaacctggntttgaagttgatgatatcagagggggtgttgctggtggcagtattttgaaggtatctttctctatttctgcaatcaatgatatgatgcaastgattgcttctttaacgttaggcttttagtttgaccaagtaatcaagtagtgcctgaggcctattagagtgtagacgtgaagatctcattggaagatgttctagataatattaattatattctttgcgcagggtgtactgaaggttaatc

SEQ ID NO: 8 - DNA, 101 bp, Solanum lycopersicum
  source 1..101: mol_type = genomic DNA
  variation 51: note = Resistant = C; Susceptible = T
agacagtagtttcagtatacaggatcaatgacctttcacaccctcaatatygcttcaaggtcgatgtcaatgcacgggagaatcacatgaccgggtgtgcg

SEQ ID NO: 9 - DNA, 101 bp, Solanum lycopersicum
  source 1..101: mol_type = genomic DNA
  variation 51: note = Resistant = G; Susceptible = T
acaggcagagaccatgttgatcttgtctgtatcgttccaattttatcgaakgtccataaagggatccgtgttctgcaaaagttgtgtactctttaatttga

SEQ ID NO: 10 - DNA, 101 bp, Solanum lycopersicum
  source 1..101: mol_type = genomic DNA
  variation 51: note = Resistant = A; Susceptible = C
cccctgattcaactagatttcttccaaatgattcttgaattagccttgaamgatgcagatgaaagatcaagttttgaagaaaatgtcatgtttgtggagaa

SEQ ID NO: 11 - DNA, 101 bp, Solanum lycopersicum
  source 1..101: mol_type = genomic DNA
  variation 51: note = Resistant = C; Susceptible = T
cttccttcacggtcaacctccgccgtactccgccatattcttcccgtcgtyagcgacttctccctgaatatcaacactcaacctaagtttccggtgaacag

(19) United States
(12) Patent Application Publication
(10) Pub. No.: US 2025/0120356 A1
(43) Pub. Date: Apr. 17, 2025

(54) METHODS AND COMPOSITIONS FOR TOMATO SPOTTED WILT VIRUS RESISTANCE IN TOMATO

(71) Applicant: Monsanto Technology LLC, St. Louis, MO (US)

(72) Inventors: Brad K. Gabor, Woodland, CA (US); Elaine Graham, Davis, CA (US); Jessica Packard Selby, St. Louis, MO (US); Rebeca Noelani Schauland, Sacramento, CA (US); Susan Anne Van Tuyl, Dixon, CA (US)

(21) Appl. No.: 18/913,504

(22) Filed: Oct. 11, 2024

Related U.S. Application Data
(60) Provisional application No. 63/590,105, filed on Oct. 13, 2023.

(51) Int. Cl. A01H 1/04; C12Q 1/6895

(52) U.S. Cl. CPC A01H 1/045; C12Q 1/6895; C12Q 2600/13

(57) ABSTRACT

Tomato (Solanum lycopersicum) plants exhibiting resistance to resistance to resistance breaking isolates of tomato spotted wilt virus (TSWV) are provided, together with methods of producing, identifying, or selecting plants or germplasm with a TSWV resistance phenotype. Such plants include tomato plants comprising introgressed genomic regions conferring pest resistance. Compositions, including novel polymorphic markers for detecting plants comprising introgressed loci, are further provided.

### CROSS-REFERENCE TO RELATED APPLICATIONS

This application claims the priority of U.S. Provisional Appl. Ser. No. 63/590,105, filed Oct. 13, 2023, the entire disclosure of which is incorporated herein by reference.

### FIELD OF THE INVENTION

The present invention relates to the field of plant breeding and, more specifically, to methods and compositions for producing tomato plants with desired resistance to tomato spotted wilt virus (TSWV).

### INCORPORATION OF SEQUENCE LISTING

The sequence listing that is contained in the file named “SEMB051US_ST26.xml,” which is 16.1 kilobytes as measured in Microsoft Windows operating system and was created on Oct. 10, 2024, is filed electronically herewith and incorporated herein by reference.

### BACKGROUND OF THE INVENTION

Plant disease resistance is an important trait in plant breeding, particularly for production of food crops. Tomato spotted wilt virus (TSWV) is a Tospovirus that causes major economic losses as it affects numerous agronomic species worldwide. In tomatoes, TSWV symptoms include chlorosis, necrosis, wilting, stunting and even death. Infection can significantly reduce yields and render fruits unmarketable. The virus is difficult to control as it is transmitted by multiple thrips species and has a wide host range (>1000 species). Furthermore, breaking isolates that overcome resistance conferred by known TSWV resistance sources have been reported in multiple countries around the world. The ability of these breaking isolates to overcome resistance in tomato plants presents a significant threat to tomato production. The development of tomato varieties with effective levels of resistance to TSWV is therefore increasingly important.

### SUMMARY OF THE INVENTION

In one aspect, the invention provides a Solanum lycopersicum plant comprising a recombinant chromosomal segment on chromosome 2, a recombinant chromosomal segment on chromosome 5, or a recombinant chromosomal segment on both chromosome 2 and 5, wherein each of said chromosomal segments comprises a tomato spotted wilt virus (TSWV) resistance allele from Solanum pimpinellifolium conferring increased resistance to resistance breaking isolates relative to a plant lacking said chromosomal segment(s). In some embodiments, the TSWV resistance allele from Solanum pimpinellifolium is located on chromosome 2; or the TSWV resistance allele from Solanum pimpinellifolium is located on chromosome 5. In certain embodiments, said plant comprises both TSWV resistance alleles from Solanum pimpinellifolium on chromosome 2 and chromosome 5. In other embodiments, said TSWV resistance allele on chromosome 2 is located within a chromosomal segment proximal to M4 (SEQ ID NO:4) in said plant. In further embodiments, said recombinant chromosomal segment on chromosome 2 is located between 1 bp and 31,168,278 bp of public tomato genome sequence SL5.0. In some embodiments, said TSWV resistance allele on chromosome 5 is located within a chromosomal segment distal to M11 (SEQ ID NO:11). In further embodiments, said recombinant chromosomal segment on chromosome 5 is located between 64,287,180 bp and 65,608,067 bp of public tomato genome sequence SL5.0. In certain embodiments, said recombinant chromosomal segment on chromosome 2 comprises a marker locus selected from the group consisting of marker locus M1 (SEQ ID NO:1), M2 (SEQ ID NO:2), M3 (SEQ ID NO:3), and M4 (SEQ ID NO:4). In some embodiments, said recombinant chromosomal segment on chromosome 5 comprises a marker locus selected from the group consisting of marker locus M8 (SEQ ID NO:8), M9 (SEQ ID NO:9), M10 (SEQ ID NO:10), and M11 (SEQ ID NO:11). In some embodiments, said TSWV resistance allele confers resistance to Sw-5b resistance breaking TSWV isolates. In other embodiments, either or both of said TSWV resistance alleles in said plant are in homozygous or heterozygous form.

In another aspect, cells, seed, and plant parts comprising a recombinant chromosomal segment on chromosome 2, a recombinant chromosomal segment on chromosome 5, or both, wherein each of said chromosomal segments comprises a tomato spotted wilt virus (TSWV) resistance alleles from Solanum pimpinellifolium conferring increased resistance to resistance breaking isolates relative to a plant lacking said chromosomal segment(s) are provided. In certain embodiments, a representative sample of seed comprising each of said chromosomal segments was deposited under NCMA Accession Number 202302005. Cells, seeds, and plant parts comprising one or both chromosomal segments are further provided.

In another aspect, methods are provided for producing a Solanum lycopersicum plant with resistance to resistance breaking tomato spotted wilt virus (TSWV) isolates, comprising introgressing into said plant a chromosomal segment from Solanum pimpinellifolium on chromosome 2, a chromosomal segment from Solanum pimpinellifolium on chromosome 5, or both, that confer resistance to tomato spotted wilt virus (TSWV) relative to a plant lacking said recombinant chromosomal segment(s). In some embodiments, said introgressing comprises crossing a plant comprising said chromosomal segment(s) with itself or with a second Solanum lycopersicum plant of a different genotype to produce one or more progeny plants; and selecting a progeny plant comprising either or both of said chromosomal segments. In other embodiments, selecting said progeny plant comprises detecting at least one allele proximal to M4 (SEQ ID NO:4) on chromosome 2; or distal to M11 (SEQ ID NO:11) on chromosome 5. In further embodiments, selecting a progeny plant comprises detecting nucleic acids comprising marker locus M1 (SEQ ID NO:1), M2 (SEQ ID NO:2), M3 (SEQ ID NO:3), M4 (SEQ ID NO:4), or M5 (SEQ ID NO:5). In some embodiments, selecting a progeny plant comprises detecting nucleic acids comprising marker locus M7 (SEQ ID NO:7), M8 (SEQ ID NO:8), M9 (SEQ ID NO:9), M10 (SEQ ID NO:10), or M11 (SEQ ID NO:11). In other embodiments, crossing a plant comprising one or both chromosomal segments comprises backcrossing, marker-assisted selection, or assaying for said TSWV resistance. In certain embodiments, the resistance comprises resistance to Sw-5b resistance breaking TSWV isolates. In yet further embodiments, the progeny plant is an F₂-F₆ progeny plant or producing said progeny plant comprises backcrossing.

In further aspects, methods are provided for producing a Solanum lycopersicum plant with resistance to resistance breaking tomato spotted wilt virus (TSWV) isolates, comprising introgressing into a plant an allele from Solanum pimpinellifolium conferring resistance to resistance breaking tomato spotted wilt virus (TSWV) isolates, wherein said resistance allele is defined as located in a genomic region proximal to M4 (SEQ ID NO:4) on chromosome 2; or distal to M11 (SEQ ID NO:11) on chromosome 5. In certain embodiments, said genomic region is proximal to M4 (SEQ ID NO:4) on chromosome 2; or said genomic region is distal to M11 (SEQ ID NO:11) on chromosome 5. Said introgressing may comprise backcrossing, marker-assisted selection, or assaying for said TSWV resistance. Tomato plants obtainable by the methods disclosed herein are further provided.

In yet a further aspect, methods are provided for selecting a Solanum lycopersicum plant exhibiting resistance to resistance breaking tomato spotted wilt virus (TSWV) isolates, comprising: a) screening one or more plants with at least one nucleic acid marker to detect a polymorphism genetically linked to said TSWV resistance; and b) selecting one or more plants comprising said polymorphism genetically linked to said TSWV resistance, wherein said polymorphism is within or genetically linked to a chromosomal segment proximal to M4 (SEQ ID NO:4) in said plant on chromosome 2; or a chromosomal segment distal to M11 (SEQ ID NO:11) in said plant on chromosome 5. In some embodiments, selecting said progeny plant comprises detecting at least one polymorphism at a locus selected from the group consisting of marker locus M1 (SEQ ID NO:1), M2 (SEQ ID NO:2), M3 (SEQ ID NO:3), M4 (SEQ ID NO:4), and M5 (SEQ ID NO:5). In other embodiments, selecting said progeny plant comprises detecting at least one polymorphism at a locus selected from the group consisting of marker locus M6 (SEQ ID NO:6), M7 (SEQ ID NO:7), M8 (SEQ ID NO:8), M9 (SEQ ID NO:9), M10 (SEQ ID NO:10), and M11 (SEQ ID NO:11). In further embodiments, screening one or more plants comprises PCR, single strand conformational polymorphism analysis, denaturing gradient gel electrophoresis, cleavage fragment length polymorphism analysis, TAQMAN assay, and/or DNA sequencing. In yet further embodiments, the Solanum lycopersicum plant comprises said chromosomal segment on chromosome 2, said recombinant chromosomal segment on chromosome 5, or said chromosomal segments on both chromosome 2 and 5, and wherein a representative sample of seed comprising each of said chromosomal segments has been deposited under NCMA Accession No. 202302005.

### BRIEF DESCRIPTION OF THE DRAWINGS

FIG. 1: From rough mapping, the mean disease scores of F₂ tomato plants are shown with varying genotypic combinations at two markers identified in rough mapping as associated with the QTLs located on chromosomes 2 and 5 (M2 and M6 respectively). The symbols “+” and “−” indicate the presence of the resistance (SPRJ2018) and susceptible (P1) alleles. The x-axis gives the genotypes on chromosome 5 for marker M6: “−/−” homozygous susceptible, “+/−” heterozygous; and “+/+” homozygous, and the three different line pattern (continuous, dashed or doted) correspond to the three different genotypes on chromosome 2 for marker M2 (see figure legend).

FIG. 2: Shows an overview of marker positions and chromosomal region on chromosome 2. The shaded rectangle indicates the chromosomal fragment that confers resistance to tomato spotted wilt virus breaking isolates in tomato. The black circle indicates the estimated position of the centromere. The QTL interval overlaps with the centromere, which is a region of reduced recombination, and this is reflected in the difference between the genetic and the physical sizes of the interval.

FIG. 3: Shows an overview of marker positions and chromosomal region on chromosome 5. The shaded rectangle indicates the chromosomal fragment that confers resistance to tomato spotted wilt virus breaking isolates in tomato. The circle indicates the estimated position of the centromere.

FIG. 4: Shows the effect of different genotypic combinations of QTL2 and QTL5 on resistance to rbTSWV in three different elite Solanum lycopersicum backgrounds. BC₁F₃ populations were created between the elite lines and the resistant donor, SPRJ2018, and plants containing the two-locus (chr2/chr5) genotypes shown on the x-axis were selected and tested for resistance. For example, the genotype “het/hmz” means these plants were heterozygous for the SPRJ2018 donor haplotype across the full QTL2 interval and homozygous for the SPRJ2018 haplotype across the full QTL5 interval. The mean resistance levels of genotypes connected by the same letters are not significantly different based on a Games-Howell post-hoc analysis. For example, the section denoted by “a” indicates that BC₁F₃ plants lacking both QTLs were not significantly different from the susceptible elite lines.

### DETAILED DESCRIPTION OF THE INVENTION

Tomato spotted wilt virus (TSWV) is a devastating disease for many different crops grown in temperate and subtropical regions of the world. The host range for TSWV is one of the widest known for plant viruses, and infects over 1,000 species in 85 families, including both monocots and dicots. The virus has been confirmed in begonia, cowpea, impatiens, peanut, pepper, potato, squash, and tomato. In addition to tomato, other susceptible plants include celery, cucumber, eggplant, lettuce, onion, peppermint, spinach, watermelon, many legumes, and many ornamentals. Symptoms of TSWV can vary, but typically include young leaves turning bronze and subsequently developing numerous small, dark spots; and leaves often drooping on the plant, creating a wilt-like appearance. Other symptoms include dieback of the growing tips, stunting, mottling, and dark streaking of the terminal stems. Affected plants may develop a one-sided growth habit or may be stunted completely. Plants that are affected early in the growing season often do not produce any fruit, while those infected after fruit set produce fruit with striking symptoms, including chlorotic concentric ring spots, raised bumps, uneven ripening, and deformation.

TSWV is transmitted from infected plants to healthy plants by at least ten species of thrips, which transmit the virus in a persistent, propagative manner. Thus, host resistance is the most effective method of managing TSWV. A number of different TSWV resistance genes have been described in tomato. These include Sw-1a, Sw-1b, sw-2, sw-3, sw-4, Sw-5b, Sw-6, and Sw-7. The loci Sw-1 through Sw-4 have been named and generally described; however, none of these have been characterized genetically or mapped (Finlay, Aust. J. Biol. Sci. 6:153-163, 1953). Only Sw-5b (from Solanum peruvianum) and Sw-7 (from Solanum chilense accession LA 1938) have been mapped to chromosomes 9 and 12, respectively (Stevens et. al., Theor. Appl. Genet. 90:445-456, 1995; Canady et al., Euphytica 117(1):19-25, 2001). Additional resistance alleles at the Sw-5b locus have been described including one discovered in the breeding line UPV 1 with resistance derived from Solanum peruvianum accession PE-18 (Rosello et al., Euphytica 119(3):357-367, 2001), and Sw-5b2 (Belfanti et al.; EP3082403B1) from an unknown source. The Sw-6 (UPV 32) locus was identified in Solanum peruvianum from the same accession as UPV 1 (Rosello et al., Eur. J. Plant Pathol. 104:499-509, 1998) although others were unable to confirm the resistance from this source in separate testing (Price et al., TGC Report No. 57, 35-36, 2007). More recently, a novel QTL from the resistant cultivar H149 (Solanum lycopersicum var. cerasiforme) was mapped to chromosome 5 between markers at 3.9 Mb and 4.1 Mb (Qi et al., Theor. Appl. Genet. 135(5):1493-1509, 2022).

Although multiple sources of TSWV resistance have been described, a majority of these sources provide isolate-specific resistance. The Sw-5b locus from Solanum peruvianum, however, was found to confer broad resistance to most TSWV isolates. Due to its resistance profile, the Sw-5b locus has been widely deployed in commercial cultivars. This has led to the emergence of isolates that are able to overcome the resistance conferred by Sw-5b. Such isolates have been reported in multiple countries around the world. Resistance breaking isolates present a significant threat to tomato production. Therefore, developing compositions and methods that confer robust resistance to TSWV resistance breaking isolates are of utmost importance.

The inventors identified two novel QTLs (one on chromosome 2 and one on chromosome 5) from Solanum pimpinellifolium that provide resistance to Sw-5b-breaking TSWV isolates. These newly identified resistance loci do not co-localize with any of the previously characterized resistance loci described above. The present disclosure represents a significant advance in that it provides, in one embodiment, a Solanum pimpinellifolium source (SPRJ2018) that demonstrates resistance against TSWV isolates that break the resistance conferred by the Sw-5b locus. Specifically, two novel TSWV resistance loci were identified in SPRJ2018. The first QTL is located on chromosome 2 between the start of the chromosome and 31.2 Mb; and the second QTL is located on chromosome 5 between 64.3 Mb and the end of the chromosome at 65.7 Mb, of the tomato reference genome SL5.0 (Zhou et al., Nature 606:527-534, 2022); available at phytozome-next.jgi.doe.gov/info/Slycopersicum_ITAG5_0 and solgenomics.net/ftp/genomes/TGG/genome/).

The present invention represents a significant advance in that it provides, in one embodiment, TSWV resistance in tomato plants conferred by a novel QTL on chromosome 2 and/or a novel QTL on chromosome 5 as well as novel recombinant chromosomal segments comprising the QTLs. The resistance profile and QTLs are distinct from those known in the art, with significantly increased resistance when deployed together. In addition, novel markers for the new loci are provided, allowing the loci to be accurately introgressed and tracked during development of new varieties. As such, the invention permits introgression of the disease resistance loci into potentially any desired tomato genotype.

One aspect of the present disclosure therefore provides resistance to resistance breaking TSWV isolates in tomato plants conferred by a novel recombinant chromosomal segment from Solanum pimpinellifolium comprising the QTL on chromosome 2 and/or a novel recombinant chromosomal segment from Solanum pimpinellifolium comprising the QTL on chromosome 5, as well as methods for the production thereof. Novel markers for the new loci are provided herein, allowing at least one locus, or both novel loci, to be accurately introgressed and tracked during development of new varieties. As such, the invention permits introgression of the TSWV resistance loci derived from Solanum pimpinellifolium into potentially any desired elite tomato variety.

In certain embodiments, Solanum lycopersicum plants are provided herein comprising a recombinant chromosomal segment from Solanum pimpinellifolium on chromosome 2 or a recombinant chromosomal segment from Solanum pimpinellifolium on chromosome 5, wherein the chromosomal segment comprises a tomato spotted wilt virus (TSWV) resistance allele that confers increased resistance to resistance breaking TSWV isolates relative to a plant lacking said chromosomal segment. In further embodiments, plants are provided comprising combinations of TSWV resistance loci on chromosomes 2 and 5, e.g. recombinant chromosomal segments from Solanum pimpinellifolium on chromosome 2 and 5, wherein each of the chromosomal segments comprise a tomato spotted wilt virus (TSWV) resistance allele that confers increased resistance to resistance breaking TSWV isolates relative to a plant lacking said chromosomal segments.

In some embodiments, a TSWV resistance allele from Solanum pimpinellifolium provided by the invention is located on chromosome 2, within a recombinant chromosomal segment extending from the start of the chromosome and flanked by marker M4 (SEQ ID NO:4) or marker M5 (SEQ ID NO:5). In certain embodiments, marker M1 (SEQ ID NO:1) is within or genetically linked to said recombinant chromosomal segment. Marker M1 (SEQ ID NO:1) is a SNP marker with a [T/C] change at 2,317,254 bp on chromosome 2 of the tomato reference genome SL5.0, marker M4 (SEQ ID NO:4) is a SNP marker with a [A/C] change at 31,168,278 bp of chromosome 2, and marker M5 (SEQ ID NO:5) is a SNP marker with a [C/T] change at 34,800,931 bp of chromosome 2 of the tomato reference genome SL5.0. In other embodiments, such a chromosomal segment can comprise one or more of marker M1 (SEQ ID NO:1), marker M2 (SEQ ID NO:2), marker M3 (SEQ ID NO:3), marker M4 (SEQ ID NO:4), and marker M5 (SEQ ID NO:5). Markers M2 (SEQ ID NO:2) and M3 (SEQ ID NO:3) are interstitial markers, where marker M2 (SEQ ID NO:2) is a SNP marker with a [A/C] change at 14,999,363 bp of chromosome 2 of the tomato reference genome SL5.0 and marker M3 (SEQ ID NO:3) is a SNP marker with a [A/G] change at 23,620,465 bp of chromosome 2 of the tomato reference genome SL5.0. In some embodiments, the TSWV resistance allele from Solanum pimpinellifolium may be defined as located on chromosome 2, within a recombinant chromosomal segment flanked by marker M1 and marker M5. In certain embodiments, the TSWV resistance allele from Solanum pimpinellifolium may be defined as located on chromosome 2, within a recombinant chromosomal segment flanked by marker M1 and marker M4. In further embodiments, one or both of the flanking markers are interstitial markers between M1 (SEQ ID NO:1) and M5 (SEQ ID NO:5), such as markers M2 (SEQ ID NO:2), M3 (SEQ ID NO:3), and M4 (SEQ ID NO:4).

In some embodiments, a TSWV resistance allele from Solanum pimpinellifolium provided by the invention is located on chromosome 5, within a recombinant chromosomal segment flanked by marker M11 (SEQ ID NO:11) or marker M7 (SEQ ID NO:7) and the end of the chromosome. In certain embodiments, marker M10 (SEQ ID NO:10) is within or genetically linked to said recombinant chromosomal segment. In other embodiments, marker M6 (SEQ ID NO:6) is genetically linked to said recombinant chromosomal segment. Marker M11 (SEQ ID NO:11) is a SNP marker with a [T/C] change at 64,287,180 bp of chromosome 5 of the tomato reference genome SL5.0; marker M7 (SEQ ID NO:7) is a SNP marker with a [G/C] change at 64,076,471 bp of chromosome 5; marker M10 (SEQ ID NO:10) is a SNP marker with a [C/A] change at 65,608,067 bp of chromosome 5 of the tomato reference genome SL5.0; and marker M6 (SEQ ID NO:6) is a SNP marker with a [T/C] change at 62,940,825 bp of chromosome 5. In other embodiments, such a chromosomal segment can comprise one or more of marker M11 (SEQ ID NO:11), marker M8 (SEQ ID NO:8), marker M9 (SEQ ID NO:9), and marker M10 (SEQ ID NO:10). Markers M8 (SEQ ID NO:8) and M9 (SEQ ID NO:9) are interstitial markers, where marker M8 (SEQ ID NO:8) is a SNP marker with a [T/C] change at 64,505,710 bp of chromosome 5 of the tomato reference genome SL5.0 and marker M9 (SEQ ID NO:9) is a SNP marker with a [T/G] change at 65,177,794 bp of chromosome 5 of the tomato reference genome SL5.0. In some embodiments, the TSWV resistance allele from Solanum pimpinellifolium may be defined as located on chromosome 5, within a recombinant chromosomal segment flanked by marker M7 and marker M10. In certain embodiments, the TSWV resistance allele from Solanum pimpinellifolium may be defined as located on chromosome 5, within a recombinant chromosomal segment flanked by marker M11 and marker M10. In certain embodiments, one or both of the flanking markers are interstitial markers between M7 (SEQ ID NO:7) and M10 (SEQ ID NO:10), such as markers M11 (SEQ ID NO:11), M8 (SEQ ID NO:8), and M9 (SEQ ID NO:9).

The tomato reference genome is publicly available, for example, www.solgenomics.net, and one skilled in the art would understand that the marker sequences provided for the first time in the instant application could be located on any version (or later version) of the public genome.

In certain embodiments, the invention provides methods of producing or selecting a tomato plant exhibiting resistance to tomato spotted wilt virus (TSWV) comprising a) crossing a tomato plant provided herein with itself or with a second tomato plant of a different genotype to produce one or more progeny plants; and b) selecting a progeny plant comprising a TSWV resistance allele.

In some embodiments, methods of the invention comprise selecting a progeny plant by detecting nucleic acids comprising marker locus M1 (SEQ ID NO:1), marker locus M2 (SEQ ID NO:2), marker locus M3 (SEQ ID NO:3), marker locus M4 (SEQ ID NO: 4), marker locus M5 (SEQ ID NO:5) on chromosome 2, or marker locus M6 (SEQ ID NO:6), marker locus M7 (SEQ ID NO:7), marker locus M8 (SEQ ID NO:8), marker locus M9 (SEQ ID NO:9), marker locus M10 (SEQ ID NO:10), or M11 (SEQ ID NO:11) on chromosome 5. In particular embodiments, selecting a tomato plant exhibiting resistance to resistance breaking TSWV comprises molecular genetic techniques. For example, those of ordinary skill in the art viewing the present disclosure may use technical methods to select a tomato plant exhibiting resistance to resistance breaking TSWV by screening one or more plants with at least one nucleic acid marker to detect a polymorphism genetically linked to TSWV resistance.

Because genetically diverse plant lines can be difficult to cross, the introgression of TSWV resistance loci and/or alleles into cultivated lines using conventional breeding methods could require prohibitively large segregating populations for progeny screens with an uncertain outcome. Marker-assisted selection (MAS) is therefore essential for the effective introgression of loci that confer resistance to TSWV into elite cultivars. For the first time, the present disclosure enables effective MAS by providing improved and validated markers for detecting genotypes associated with TSWV resistance without the need to grow large populations of plants to maturity in order to observe the phenotype.

### I. Genomic Region, QTL, Polymorphic Nucleic Acids, and Alleles Associated with TSWV Resistance

The invention provides novel introgressions of one or more loci associated with resistance to TSWV in tomato, together with polymorphic nucleic acids and linked markers for tracking the introgressions during plant breeding. The seeds deposited under NCMA Accession No. 202302005 may be used as a source for the recombinant chromosomal segments on chromosomes 2 and 5.

Using the improved genetic markers and assays of the invention, the present inventors were able to successfully identify novel introgressions that confers to a tomato plant resistance to TSWV. In certain embodiments, the invention provides tomato plants comprising donor DNA between the start of chromosome 2 and marker locus M4 (SEQ ID NO:4) on chromosome 2; or donor DNA between marker locus M11 (SEQ ID NO:11) and the end of the chromosome on chromosome 5. In some embodiments, the invention provides tomato plants comprising the donor DNA on both chromosome 2 and chromosome 5. In specific embodiments, the donor DNA is genetically linked to marker M1 (SEQ ID NO:1) on chromosome 2; or genetically linked to marker M10 (SEQ ID NO:10) on chromosome 5.

### II. Introgression of a Genomic Locus Associated with Resistance to TSWV in Tomato

Marker-assisted introgression involves the transfer of a chromosomal region defined by one or more markers from a first genetic background to a second. Offspring of a cross that contain the introgressed genomic region can be identified by the combination of markers characteristic of the desired introgressed genomic region from a first genetic background and both linked and unlinked markers characteristic of the second genetic background.

Provided herein are novel accurate markers for identifying and tracking introgression of one or more of the genomic regions disclosed herein from a TSWV resistant plant into a cultivated line. The invention further provides markers for identifying and tracking the novel introgressions disclosed herein during plant breeding, including the markers set forth in Table 1. Markers within or linked to any of the genomic intervals of the present disclosure may be useful in a variety of breeding efforts that include introgression of genomic regions associated with TSWV resistance into a desired genetic background. For example, a marker within 40 cM, 20 cM, 15 cM, 10 cM, 5 cM, 2 cM, or 1 cM of a marker associated with TSWV resistance described herein can be used for marker-assisted introgression of genomic regions associated with a TSWV resistant phenotype.

Markers that are linked and either immediately adjacent or adjacent to the identified TSWV resistance QTL that permit introgression of the locus in the absence of extraneous linked DNA from the source germplasm containing the QTL are provided herewith. Those of skill in the art will appreciate that when seeking to introgress a smaller genomic region comprising a QTL associated with resistance to TSWV described herein, that any of the telomere proximal or centromere proximal markers that are immediately adjacent to a larger genomic region comprising the QTL can be used to introgress that smaller genomic region.

Tomato plants or germplasm comprising an introgressed region that is associated with resistance to TSWV wherein at least 10%, 25%, 50%, 75%, 90%, or 99% of the remaining genomic sequences carry markers characteristic of plant or germplasm that otherwise or ordinarily comprise a genomic region associated with another phenotype, are thus provided in specific embodiments. Furthermore, tomato plants comprising an introgressed region where closely linked regions adjacent and/or immediately adjacent to the genomic regions, QTL, and markers provided herewith that comprise genomic sequences carrying markers characteristic of tomato plants or germplasm that otherwise or ordinarily comprise a genomic region associated with the phenotype are also provided.

### III. Development of Tomato Plants with Resistance to TSWV

For most breeding objectives, commercial breeders may work within germplasm that is often referred to as the “cultivated” or “elite.” This germplasm is easier to use in plant breeding because it generally performs well when evaluated for horticultural performance. The performance advantage a cultivated variety provides is sometimes offset by a lack of allelic diversity. Breeders generally accept this tradeoff because progress is faster when working with cultivated material than when breeding with genetically diverse sources.

In contrast, when cultivated germplasm is crossed with non-cultivated germplasm, a breeder can gain access to novel alleles from the non-cultivated type. However, this approach presents significant difficulties due to fertility problems associated with crosses between diverse lines, and negative linkage drag from the non-cultivated parent. In tomato plants, non-cultivated types such as Solanum pimpinellifolium can provide alleles associated with disease resistance. However, these non-cultivated types may have poor horticultural qualities.

The process of introgressing desirable resistance genes from non-cultivated lines into elite cultivated lines while avoiding problems with genetically linked deleterious loci or low heritability is a long and often arduous process. In deploying loci derived from wild relatives it is often desirable to introduce a minimal or truncated introgression that provides the desired trait but lacks detrimental effects. To aid introgression reliable marker assays are preferable to phenotypic screens. Success is furthered by simplifying genetics for key attributes to allow focus on genetic gain for quantitative traits such as TSWV resistance. Moreover, the process of introgressing genomic regions from non-cultivated lines can be greatly facilitated by the availability of accurate markers for MAS.

One of skill in the art would therefore understand that the loci, polymorphisms, and markers provided by the invention allow the tracking and introduction of any of the genomic regions identified herein into any genetic background. In addition, the genomic regions associated with TSWV resistance disclosed herein can be introgressed from one genotype to another and tracked using MAS. Thus, the inventors' discovery of accurate markers associated with TSWV resistance will facilitate the development of tomato plants having beneficial phenotypes. For example, seed can be genotyped using the markers of the present disclosure to select for plants comprising desired genomic regions associated with TSWV resistance. Moreover, MAS allows identification of plants homozygous or heterozygous for a desired introgression.

Inter-species crosses can also result in suppressed recombination and plants with low fertility or fecundity. For example, suppressed recombination has been observed for the tomato nematode resistance gene Mi, the Mla and Mlg genes in barley, the Yr17 and Lr20 genes in wheat, the Run1 gene in grapevine, and the Rma gene in peanut. Meiotic recombination is essential for classical breeding because it enables the transfer of favorable loci across genetic backgrounds, the removal of deleterious genomic fragments, and pyramiding traits that are genetically tightly linked. Therefore, suppressed recombination forces breeders to enlarge segregating populations for progeny screens in order to arrive at the desired genetic combination.

Phenotypic evaluation of large populations is time-consuming, resource-intensive and not reproducible in every environment. Marker-assisted selection offers a feasible alternative. Molecular assays designed to detect unique polymorphisms, such as SNPs, are versatile. However, they may fail to discriminate loci within and among tomato species in a single assay. Structural rearrangements of chromosomes such as deletions impair hybridization and extension of synthetically labeled oligonucleotides. In the case of duplication events, multiple copies are amplified in a single reaction without distinction. The development and validation of accurate and highly predictive markers are therefore essential for successful MAS breeding programs.

### IV. Marker-Assisted Breeding and Genetic Engineering Techniques

Genetic markers that can be used in the practice of the present invention include, but are not limited to, restriction fragment length polymorphisms (RFLPs), amplified fragment length polymorphisms (AFLPs), simple sequence repeats (SSRs), simple sequence length polymorphisms (SSLPs), single nucleotide polymorphisms (SNPs), insertion/deletion polymorphisms (Indels), variable number tandem repeats (VNTRs), and random amplified polymorphic DNA (RAPD), isozymes, and other markers known to those skilled in the art. Marker discovery and development in crop plants provides the initial framework for applications to marker-assisted breeding activities (U.S. Patent Pub. Nos. 2005/0204780; 2005/0216545; 2005/0218305; and 2006/00504538). The resulting “genetic map” is the representation of the relative position of characterized loci (polymorphic nucleic acid markers or any other locus for which loci can be identified) to each other.

Polymorphisms comprising as little as a single nucleotide change can be assayed in a number of ways. For example, detection can be made by electrophoretic techniques including a single strand conformational polymorphism (Orita et al., Genomics 8(2):271-278, 1989), denaturing gradient gel electrophoresis (Myers, EP0273085), or cleavage fragment length polymorphisms (Life Technologies, Inc., Gaithersburg, MD), but the widespread availability of DNA sequencing often makes it easier to simply sequence amplified products directly. Once the polymorphic sequence difference is known, rapid assays can be designed for progeny testing, typically involving some version of PCR amplification of specific loci (PASA; Sommer et al., Biotechniques 12(1):82-87, 1992), or PCR amplification of multiple specific loci (PAMSA; Dutton and Sommer, Biotechniques 11(6):700-7002, 1991).

Polymorphic markers serve as useful tools for assaying plants for determining the degree of identity of lines or varieties (U.S. Pat. No. 6,207,367). These markers form the basis for determining associations with phenotypes and can be used to drive genetic gain. In certain embodiments of methods of the invention, polymorphic nucleic acids can be used to detect in a tomato plant a genotype associated with TSWV resistance, identify a tomato plant with a genotype associated with TSWV resistance, and to select a tomato plant with a genotype associated with TSWV resistance. In certain embodiments of methods of the invention, polymorphic nucleic acids can be used to produce a tomato plant that comprises in its genome an introgressed locus associated with TSWV resistance. In certain embodiments of the invention, polymorphic nucleic acids can be used to breed progeny tomato plants comprising a locus or loci associated with TSWV resistance.

Genetic markers may include “dominant” or “codominant” markers. “Codominant” markers reveal the presence of two or more loci (two per diploid individual). “Dominant” markers reveal the presence of only a single locus. Markers are preferably inherited in codominant fashion so that the presence of both loci at a diploid locus, or multiple loci in triploid or tetraploid loci, are readily detectable, and they are free of environmental variation, i.e., their heritability is 1. A marker genotype typically comprises two marker loci at each locus in a diploid organism. The marker allelic composition of each locus can be either homozygous or heterozygous. Homozygosity is a condition where both loci at a locus are characterized by the same nucleotide sequence. Heterozygosity refers to a condition where the two loci at a locus are different.

Nucleic acid-based analyses for determining the presence or absence of the genetic polymorphism (i.e. for genotyping) can be used in breeding programs for identification, selection, introgression, and the like. A wide variety of genetic markers for the analysis of genetic polymorphisms are available and known to those of skill in the art. The analysis may be used to select for genes, portions of genes, QTL, loci, or genomic regions that comprise or are linked to a genetic marker that is linked to or associated with TSWV resistance in tomato plants.

As used herein, nucleic acid analysis methods include, but are not limited to, PCR-based detection methods (for example, TaqMan assays), microarray methods, mass spectrometry-based methods and/or nucleic acid sequencing methods, including whole genome sequencing. In certain embodiments, the detection of polymorphic sites in a sample of DNA, RNA, or cDNA may be facilitated through the use of nucleic acid amplification methods. Such methods specifically increase the concentration of polynucleotides that span the polymorphic site, or include that site and sequences located either distal or proximal to it. Such amplified molecules can be readily detected by gel electrophoresis, fluorescence detection methods, or other means.

One method of achieving such amplification employs the polymerase chain reaction (PCR) (Mullis et al., Cold Spring Harbor Symp. Quant. Biol. 51:263-273, 1986; European Patent No. 50,424; European Patent No. 84,796; European Patent No. 258,017; European Patent No. 237,362; European Patent No. 201,184; U.S. Pat. Nos. 4,683,202; 4,582,788; and 4,683,194), using primer pairs that are capable of hybridizing to the proximal sequences that define a polymorphism in its double-stranded form. Methods for typing DNA based on mass spectrometry can also be used. Such methods are disclosed in U.S. Pat. Nos. 6,613,509 and 6,503,710, and references found therein.

Polymorphisms in DNA sequences can be detected or typed by a variety of effective methods well known in the art including, but not limited to, those disclosed in U.S. Pat. Nos. 5,468,613, 5,217,863; 5,210,015; 5,876,930; 6,030,787; 6,004,744; 6,013,431; 5,595,890; 5,762,876; 5,945,283; 5,468,613; 6,090,558; 5,800,944; 5,616,464; 7,312,039; 7,238,476; 7,297,485; 7,282,355; 7,270,981; and 7,250,252 all of which are incorporated herein by reference in their entirety. However, the compositions and methods provided herein can be used in conjunction with any polymorphism typing method to detect polymorphisms in genomic DNA samples. These genomic DNA samples used include but are not limited to, genomic DNA isolated directly from a plant, cloned genomic DNA, or amplified genomic DNA.

For instance, polymorphisms in DNA sequences can be detected by hybridization to locus-specific oligonucleotide (ASO) probes as disclosed in U.S. Pat. Nos. 5,468,613 and 5,217,863. U.S. Pat. No. 5,468,613 discloses locus specific oligonucleotide hybridizations where single or multiple nucleotide variations in nucleic acid sequence can be detected in nucleic acids by a process in which the sequence containing the nucleotide variation is amplified, spotted on a membrane and treated with a labeled sequence-specific oligonucleotide probe.

Target nucleic acid sequence can also be detected by probe ligation methods, for example as disclosed in U.S. Pat. No. 5,800,944 where sequence of interest is amplified and hybridized to probes followed by ligation to detect a labeled part of the probe.

Microarrays can also be used for polymorphism detection, wherein oligonucleotide probe sets are assembled in an overlapping fashion to represent a single sequence such that a difference in the target sequence at one point would result in partial probe hybridization (Borevitz et al., Genome Res. 13:513-523, 2003; Cui et al., Bioinformatics 21:3852-3858, 2005). On any one microarray, it is expected there will be a plurality of target sequences, which may represent genes and/or noncoding regions wherein each target sequence is represented by a series of overlapping oligonucleotides, rather than by a single probe. This platform provides for high throughput screening of a plurality of polymorphisms. Typing of target sequences by microarray-based methods is described in U.S. Pat. Nos. 6,799,122; 6,913,879; and 6,996,476.

Other methods for detecting SNPs and Indels include single base extension (SBE) methods. Examples of SBE methods include, but are not limited, to those disclosed in U.S. Pat. Nos. 6,004,744; 6,013,431; 5,595,890; 5,762,876; and 5,945,283.

In another method for detecting polymorphisms, SNPs and Indels can be detected by methods disclosed in U.S. Pat. Nos. 5,210,015; 5,876,930; and 6,030,787 in which an oligonucleotide probe having a 5′ fluorescent reporter dye and a 3′ quencher dye covalently linked to the 5′ and 3′ ends of the probe. When the probe is intact, the proximity of the reporter dye to the quencher dye results in the suppression of the reporter dye fluorescence, e.g. by Forster-type energy transfer. During PCR, forward and reverse primers hybridize to a specific sequence of the target DNA flanking a polymorphism while the hybridization probe hybridizes to polymorphism-containing sequence within the amplified PCR product. In the subsequent PCR cycle DNA polymerase with 5′→3′ exonuclease activity cleaves the probe and separates the reporter dye from the quencher dye resulting in increased fluorescence of the reporter.

In another embodiment, a locus or loci of interest can be directly sequenced using nucleic acid sequencing technologies. Methods for nucleic acid sequencing are known in the art and include technologies provided by 454 Life Sciences (Branford, CT), Agencourt Bioscience (Beverly, MA), Applied Biosystems (Foster City, CA), LI-COR Biosciences (Lincoln, NE), NimbleGen Systems (Madison, WI), Illumina (San Diego, CA), and VisiGen Biotechnologies (Houston, TX). Such nucleic acid sequencing technologies comprise formats such as parallel bead arrays, sequencing by ligation, capillary electrophoresis, electronic microchips, “biochips,” microarrays, parallel microchips, and single-molecule arrays.

Various genetic engineering technologies have been developed and may be used by those of skill in the art to introduce traits in plants. In certain aspects of the claimed invention, traits are introduced into tomato plants via altering or introducing a single genetic locus or transgene into the genome of a variety or progenitor thereof. Methods of genetic engineering to modify, delete, or insert genes and polynucleotides into the genomic DNA of plants are well-known in the art.

In specific embodiments of the invention, improved tomato lines can be created through the site-specific modification of a plant genome. Methods of genetic engineering include, for example, utilizing sequence-specific nucleases such as zinc-finger nucleases (see, for example, U.S. Patent Pub. No. 2011/0203012); engineered or native meganucleases; TALE-endonucleases (see, for example, U.S. Pat. Nos. 8,586,363 and 9,181,535); and RNA-guided endonucleases, such as those of the CRISPR/Cas systems (see, for example, U.S. Pat. Nos. 8,697,359 and 8,771,945 and U.S. Patent Pub. No. 2014/0068797). One embodiment of the invention thus relates to utilizing a nuclease or any associated protein to carry out genome modification. This nuclease could be provided heterologously within donor template DNA for templated-genomic editing or in a separate molecule or vector. A recombinant DNA construct may also comprise a sequence encoding one or more guide RNAs to direct the nuclease to the site within the plant genome to be modified. Further methods for altering or introducing a single genetic locus include, for example, utilizing single-stranded oligonucleotides to introduce base pair modifications in a plant genome (see, for example, Sauer et al., Plant Physiol. 170(4):1917-1928, 2016).

Methods for site-directed alteration or introduction of a single genetic locus are well-known in the art and include those that utilize sequence-specific nucleases, such as the aforementioned, or complexes of proteins and guide-RNA that cut genomic DNA to produce a double-strand break (DSB) or nick at a genetic locus. As is well-understood in the art, during the process of repairing the DSB or nick introduced by the nuclease enzyme, a donor template, transgene, or expression cassette polynucleotide may become integrated into the genome at the site of the DSB or nick. The presence of homology arms in the DNA to be integrated may promote the adoption and targeting of the insertion sequence into the plant genome during the repair process through homologous recombination or non-homologous end joining (NHEJ).

In another embodiment of the invention, genetic transformation may be used to insert a selected transgene into a plant of the invention or may, alternatively, be used for the preparation of transgenes which can be introduced by backcrossing. Methods for the transformation of plants that are well-known to those of skill in the art and applicable to many crop species include, but are not limited to, electroporation, microprojectile bombardment, Agrobacterium-mediated transformation, and direct DNA uptake by protoplasts.

To effect transformation by electroporation, one may employ either friable tissues, such as a suspension culture of cells or embryogenic callus or alternatively one may transform immature embryos or other organized tissue directly. In this technique, one would partially degrade the cell walls of the chosen cells by exposing them to pectin-degrading enzymes (pectolyases) or mechanically wound tissues in a controlled manner.

An efficient method for delivering transforming DNA segments to plant cells is microprojectile bombardment. In this method, particles are coated with nucleic acids and delivered into cells by a propelling force. Exemplary particles include those comprised of tungsten, platinum, and preferably, gold. For the bombardment, cells in suspension are concentrated on filters or solid culture medium. Alternatively, immature embryos or other target cells may be arranged on solid culture medium. The cells to be bombarded are positioned at an appropriate distance below the macroprojectile stopping plate.

An illustrative embodiment of a method for delivering DNA into plant cells by acceleration is the Biolistics Particle Delivery System, which can be used to propel particles coated with DNA or cells through a screen, such as a stainless steel or Nytex screen, onto a surface covered with target cells. The screen disperses the particles so that they are not delivered to the recipient cells in large aggregates. Microprojectile bombardment techniques are widely applicable and may be used to transform virtually any plant species.

Agrobacterium-mediated transfer is another widely applicable system for introducing gene loci into plant cells. An advantage of the technique is that DNA can be introduced into whole plant tissues, thereby bypassing the need for regeneration of an intact plant from a protoplast. Modern Agrobacterium transformation vectors are capable of replication in E. coli as well as Agrobacterium, allowing for convenient manipulations (Klee et al., Nat. Biotechnol. 3(7):637-642, 1985). Moreover, recent technological advances in vectors for Agrobacterium-mediated gene transfer have improved the arrangement of genes and restriction sites in the vectors to facilitate the construction of vectors capable of expressing various polypeptide coding genes. The vectors described have convenient multi-linker regions flanked by a promoter and a polyadenylation site for direct expression of inserted polypeptide coding genes. Additionally, Agrobacterium containing both armed and disarmed Ti genes can be used for transformation.

In those plant strains where Agrobacterium-mediated transformation is efficient, it is the method of choice because of the facile and defined nature of the gene locus transfer. The use of Agrobacterium-mediated plant integrating vectors to introduce DNA into plant cells is well known in the art (Fraley et al., Nat. Biotechnol. 3:629-635, 1985; U.S. Pat. No. 5,563,055).

Transformation of plant protoplasts also can be achieved using methods based on calcium phosphate precipitation, polyethylene glycol treatment, electroporation, and combinations of these treatments (see, for example, Potrykus et al., Mol. Gen. Genet. 199:183-188, 1985; Omirulleh et al., Plant Mol. Biol. 21(3):415-428, 1993; Fromm et al., Nature 312:791-793, 1986; Uchimiya et al., Mol. Gen. Genet. 204:204, 1986; Marcotte et al., Nature 335:454, 1988). Transformation of plants and expression of foreign genetic elements is exemplified in Choi et al. (Plant Cell Rep. 13:344-348, 1994), and Ellul et al. (Theor. Appl. Genet. 107:462-469, 2003).

### V. Definitions

The following definitions are provided to better define the present invention and to guide those of ordinary skill in the art in the practice of the present invention. Unless otherwise noted, terms are to be understood according to conventional usage by those of ordinary skill in the relevant art.

The term “about” is used to indicate that a value includes the standard deviation of error for the device or method being employed to determine the value. The use of the term “or” in the claims is used to mean “and/or” unless explicitly indicated to refer to alternatives only or the alternatives are mutually exclusive, although the disclosure supports a definition that refers to only alternatives and to “and/or.” When used in conjunction with the word “comprising” or other open language in the claims, the words “a” and “an” denote “one or more,” unless specifically noted. The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps. Similarly, any plant that “comprises,” “has” or “includes” one or more traits is not limited to possessing only those one or more traits and covers other unlisted traits.

As used herein, the term “plant” includes the seed (from which the plant can be grown), the whole plant or any plant parts, such as plant organs (e.g., harvested or non-harvested leaves, etc.), plant cells, plant protoplasts, plant cell- or tissue cultures from which whole plants can be regenerated, propagating or non-propagating plant cells, plants cells which are not in tissue culture (but which are, for example, in vivo in a plant or plant part), plant callus, plant cell clumps, plant transplants, seedlings, plant cells that are intact in plants, plant clones or micro-propagations, or parts of plants (e.g., harvested tissues or organs), such as plant cuttings, vegetative propagations, embryos, pollen, ovules, flowers, leaves, heads, seeds (produced on the plant after self-fertilization or cross-fertilization), clonally propagated plants, roots, stems, stalks, root tips, grafts, parts of any of these and the like, or derivatives thereof, preferably having the same genetic make-up (or very similar genetic make-up) as the plant from which it is obtained. Also any developmental stage is included, such as seedlings, cuttings prior or after rooting, mature and/or immature plants or mature and/or immature leaves. When “seeds of a plant” are referred to, these either refer to seeds from which the plant can be grown or to seeds produced on the plant, after self-fertilization or cross-fertilization.

As used herein, the term “population” means a genetically heterogeneous collection of plants that share a common parental derivation.

As used herein, the term “plant line” is, for example, a breeding line which can be used to develop one or more varieties. “Inbred line” or “inbred parent” is a line which has been developed by selfing for several generations and which can be used as a parent to produce an F₁ hybrid variety. “Hybrid” refers to the seeds harvested from crossing one plant line or variety with another plant line or variety, and the plants or plant parts grown from said seeds.

As used herein, the term “F₁ hybrid” plant (or F₁ hybrid seed) is the generation obtained from crossing two non-isogenic inbred parent lines. Thus, F₁ hybrid seeds are seeds from which F₁ hybrid plants grow.

As used herein, the term “interspecific hybrid” refers to a hybrid produced from crossing a plant of one species, e.g., Solanum lycopersicum, with a plant of another species, e.g., Solanum pimpinellifolium.

As used herein, the terms “progeny,” “progenies,” or “descendants” as used herein, refer to offspring, or the first and all further descendants derived from (or obtainable from) a plant. Progeny may be derived by regeneration of cell culture or tissue culture, or parts of a plant, or selfing of a plant, or by producing seeds of a plant. In further embodiments, progeny may also encompass tomato plants derived from crossing of at least one tomato plant with another tomato plant of the same or another variety or (breeding) line, and/or backcrossing, and/or inserting of a locus into a plant and/or mutation. A progeny is, e.g., a first generation progeny, i.e., the progeny is directly derived from, obtained from, obtainable from or derivable from the parent plant by, e.g., traditional breeding methods (selfing and/or crossing) or regeneration. However, the term “progeny” generally encompasses further generations such as second, third, fourth, fifth, sixth, seventh or more generations, i.e., generations of plants which are derived from, obtained from, obtainable from or derivable from the former generation by, e.g., traditional breeding methods, regeneration or genetic transformation techniques. For example, a second generation progeny can be produced from a first generation progeny by any of the methods mentioned above. Also double haploid plants are progeny.

As used herein, the term “tissue culture” or “cell culture” refers to an in vitro composition comprising isolated cells of the same or a different type or a collection of such cells organized into plant tissue. Tissue cultures and cell cultures of tomato, and regeneration of tomato plants therefrom, is well known in the art and widely published.

As used herein, the term “regeneration” refers to the development of a plant from in vitro cell culture or tissue culture or vegetative propagation.

As used herein, the term “vegetative propagation,” “vegetative reproduction,” and “clonal propagation” are used interchangeably herein and refer to the method of taking part of a plant and allowing that plant part to form at least roots where plant part is, e.g., defined as or derived from (e.g., by cutting off) leaf, pollen, embryo, cotyledon, hypocotyl, cells, protoplasts, meristematic cell, root, root tip, pistil, anther, flower, shoot tip, shoot, stem, fruit, and petiole. When a whole plant is regenerated by vegetative propagation, it is also referred to as a “vegetative propagation” or a “vegetatively propagated plant.”

As used herein, the term “harvested plant material” refers to plant parts (e.g., leaves detached from the whole plant) which have been collected for further storage and/or further use. As used herein, the term “harvested seeds” refers to seeds harvested from a line or variety, e.g., produced after self-fertilization or cross-fertilization and collected. As used herein, the term “harvested leaves” refers to tomato leaves, i.e., the plant without the root system, for example substantially all (harvested) leaves.

As used herein, the terms “variety” and “cultivar” mean a group of similar plants that by their genetic pedigrees and performance can be identified from other varieties within the same species.

As used herein, “elite” or “cultivated” variety means any variety that has resulted from breeding and selection for superior agronomic performance. An “elite plant” refers to a plant belonging to an elite variety. Numerous elite varieties are available and known to those of skill in the art of tomato breeding. An “elite population” is an assortment of elite individuals or varieties that can be used to represent the state of the art in terms of agronomically superior genotypes of a given crop species, such as tomato. Similarly, an “elite germplasm” or elite strain of germplasm is an agronomically superior germplasm. An “elite tomato” or “cultivated tomato” cultivar/variety refers herein to plants of the species Solanum lycopersicum (or seeds from which the plants can be grown), and parts of such plants, bred by humans for food and having good agronomic characteristics. This includes any cultivated tomato, such as breeding lines (e.g. backcross lines, inbred lines), cultivars, and varieties (open-pollinated or hybrids). Wild tomato (i.e. not cultivated tomato) such as Solanum pimpinellifolium, or wild relatives of tomato are not encompassed by this definition.

As used herein, an “allele” refers to one of two or more alternative forms of a genomic sequence at a given locus on a chromosome. All alleles at a specific locus relate to one trait or characteristic. In a diploid cell of an organism, alleles of a given gene are located at a specific location, or locus on a chromosome. One allele is present on each chromosome of the pair of homologous chromosomes. A diploid plant species may comprise a large number of different alleles at a particular locus. These may be identical alleles of the gene (homozygous) or two different alleles (heterozygous).

As used herein, “tomato spotted wilt virus,” “tomato spotted wilt orthotospovirus,” “tomato spotted wilt tospovirus,” and “TSWV” refers to a disease of plants caused by a virus of the Tospoviridae family.

As used herein, “breaking isolate” and “resistance breaking isolate” refer to newly occurring strains of TSWV that can overcome resistance conferred by known TSWV resistance alleles, e.g. resistance conferred by Sw-5b.

As used herein, the term “locus” or “loci” refers to a specific place or places or a site on a chromosome where, for example, a gene or genetic marker is found. A “quantitative trait locus (QTL)” is a chromosomal location that encodes for at least a first allele that affects the expressivity of a phenotype.

As used herein, an “introgression fragment” or “introgression segment” or “introgression region” refers to a chromosome fragment (or chromosome part or region) which has been introduced into another plant of the same or related species by crossing or traditional breeding techniques, such as backcrossing, i.e., the introgressed fragment is the result of breeding methods referred to by the verb “to introgress” (such as backcrossing). In tomato, wild tomato, or wild relatives of tomato are used to introgress fragments of the wild genome into the genome of cultivated tomato. Such a tomato plant thus has a “genome of Solanum lycopersicum” but comprises in the genome a fragment of a wild tomato or tomato relative, i.e., an introgression fragment of a donor plant. It is understood that the term “introgression fragment” never includes a whole chromosome, but only a part of a chromosome.

As used herein, the terms “distal” and “proximal” describe the position of a chromosomal segment or an introgression segment in relation to a specific reference point on a whole chromosome, i.e. “distal” means that the interval or the segment is localized on the side of the reference point distant from the chromosome centromere, and “proximal” means that the interval or the segment is localized on the side of the reference point close to the chromosome centromere.

In some embodiments, e.g. the TSWV resistance alleles provided herein is located within a chromosomal segment proximal to M5 (SEQ ID NO:5) on chromosome 2; or distal to M7 (SEQ ID NO:7) on chromosome 5. In specific embodiments, e.g. the TSWV resistance alleles provided herein may also be defined as located within a chromosomal segment proximal to M4 (SEQ ID NO:4) on chromosome 2; or distal to M11 (SEQ ID NO:11) on chromosome 5.

A genetic element, a locus, an introgression fragment or a gene or allele conferring a trait (such as TSWV resistance) is said to be “obtainable from” or can be “obtained from” or “derivable from” or can be “derived from” or “as present in” or “as found in” a plant or seed if it can be transferred from the plant or seed in which it is present into another plant or seed in which it is not present (such as a line or variety) using traditional breeding techniques without resulting in a phenotypic change of the recipient plant apart from the addition of the trait conferred by the genetic element, locus, introgression fragment, gene, or allele. The terms are used interchangeably and the genetic element, locus, introgression fragment, gene, or allele can thus be transferred into any other genetic background lacking the trait. Not only seeds deposited and comprising the genetic element, locus, introgression fragment, gene, or allele can be used, but also progeny/descendants from such seeds which have been selected to retain the genetic element, locus, introgression fragment, gene, or allele, can be used and are encompassed herein, such as commercial varieties developed from the deposited seeds or from descendants thereof. Whether a plant comprises the same genetic element, locus, introgression fragment, gene, or allele as obtainable from the deposited seeds can be determined by the skilled person using one or more techniques known in the art, such as phenotypic assays, whole genome sequencing, molecular marker analysis, trait mapping, chromosome painting, allelism tests, and the like.

As used herein, a “marker” refers to detectable characteristic that can be used to discriminate between organisms. Examples of such characteristics include, but are not limited to, genetic markers, biochemical markers, metabolites, morphological characteristics, and agronomic characteristics.

As used herein, a “molecular marker” is a piece of DNA associated with a certain genomic or chromosomal location or single nucleotide polymorphism (SNP), which is found on the chromosome close to the gene of interest. Molecular markers can be used to identify a particular sequence of DNA, or a certain location in a genome or on a chromosome, or to identify an introgression fragment. When reference is made herein to one or more molecular markers being “detectable” by a molecular marker assay, this means of course that the plant or plant part comprises the one or more markers in its genome, as the marker would otherwise not be detectable.

As used herein, “flanking markers” or “bordering markers” are molecular markers located on the chromosome on either side of an allele or gene of interest, i.e., one marker on the right side of the allele or gene and one marker on the left side of the allele or gene.

As used herein, “closely linked marker” is a marker which is physically close enough to an allele or gene to co-segregate with the allele or gene at a high frequency, i.e., the chance of recombination taking place between the allele or gene and the marker is so small that the marker can be used to reliably select for the presence of the allele or gene in a breeding program (marker-assisted selection).

As used herein, “marker-assisted selection” or “MAS” refers to a process of using the presence of molecular markers, which are genetically and physically linked to a particular locus or to a particular chromosomal region (e.g., introgression fragment), to select plants (e.g., progeny) for the presence of the specific locus or region (e.g., introgression fragment).

As used herein, “marker assay” or “genotyping assay” refers to an assay which can be used to determine the marker genotype, e.g., the SNP genotype. For example, SNP markers can be detected using a KASP-assay or other assays known to the skilled person.

As used herein, the term “phenotype” refers to the detectable characteristics of a cell or organism that can be influenced by gene expression.

As used herein, the term “genotype” refers to the specific allelic makeup of a plant.

As used herein, a “gene” refers to a nucleic acid sequence forming a genetic and functional unit and coding for one or more sequence-related RNA and/or polypeptide molecules. A gene generally contains a coding region operably linked to appropriate regulatory sequences that regulate the expression of a gene product (e.g., a polypeptide or a functional RNA). A gene can have various sequence elements, including, but not limited to, a promoter, an untranslated region (UTR), exons, introns, and other upstream or downstream regulatory sequences.

As used herein, the term “physical distance” referring to a region between loci (e.g., between molecular markers and/or between phenotypic markers) on the same chromosome is the actual physical distance expressed in base pairs (bp), kilobase pairs (kb), or megabase pairs (Mb).

As used herein, the term “genetic distance” between loci (e.g., between molecular markers and/or between phenotypic markers) on the same chromosome is measured by frequency of crossing-over, or recombination frequency (RF) and is indicated in centimorgans (cM). One cM corresponds to a recombination frequency of 1%. If no recombinants can be found, the RF is zero and the loci are either extremely close together physically or they are identical. The further apart two loci are, the higher the RF.

As used herein, the term “introgressed,” when used in reference to a genetic locus, refers to a genetic locus that has been introduced into a new genetic background, such as through backcrossing. Introgression of a genetic locus can thus be achieved through plant breeding methods and/or by molecular genetic methods. Such molecular genetic methods include, but are not limited to, various plant transformation techniques and/or methods that provide for homologous recombination, non-homologous recombination, site-specific recombination, and/or genomic modifications that provide for locus substitution or locus conversion.

As used herein, the terms “recombinant” or “recombined” in the context of a chromosomal segment refer to recombinant DNA sequences comprising one or more genetic loci in a configuration in which they are not found in nature, for example as a result of a recombination event between homologous chromosomes during meiosis.

As used herein, the term “linked,” when used in the context of nucleic acid markers and/or genomic regions, refers to markers and/or genomic regions that are located on the same linkage group or chromosome such that they tend to segregate together at meiosis. As used herein, “tolerance locus” means a locus associated with tolerance or resistance to disease. For instance, a tolerance locus according to the present invention may, in one embodiment, control tolerance or susceptibility to TSWV.

As used herein, “tolerance” or “improved tolerance” in a plant refers to the ability of the plant to perform well, for example by maintaining yield, under disease conditions or upon pest infestations. Tolerance may also refer to the ability of a plant to maintain a plant vigor phenotype under disease conditions or under pest infestations. Tolerance is a relative term, indicating that a “tolerant” plant is more able to maintain performance compared to a different (less tolerant) plant (e.g. a different plant variety) grown in similar disease conditions or under similar pest pressure. One of skill will appreciate that plant tolerance to disease or pest conditions varies widely and can represent a spectrum of more-tolerant or less-tolerant phenotypes. However, by simple observation, one of skill can generally determine the relative tolerance of different plants, plant varieties, or plant families under disease or pest conditions, and furthermore, will also recognize the phenotypic gradations of “tolerance.”

As used herein “resistance” or “improved resistance” in a plant to disease or pest conditions is an indication that the plant is more able to reduce disease or pest burden than a non-resistant or less resistant plant. Resistance is a relative term, indicating that a “resistant” plant is more able to reduce disease burden or pest burden compared to a different (less resistant) plant (e.g., a different plant variety) grown in similar disease conditions or pest pressure. One of skill will appreciate that plant resistance to disease conditions or pest infestation varies widely and can represent a spectrum of more-resistant or less-resistant phenotypes. However, by simple observation, one of skill can generally determine the relative resistance of different plants, plant varieties, or plant families under disease conditions or pest pressure, and furthermore, will also recognize the phenotypic gradations of “resistant.” Resistance can be non-specific or “broad-spectrum” or be race-specific.

As used herein, “broad-spectrum” resistance refers to resistance against more than one pathogen species or against most races or strains of the same species. “Species-nonspecific” broad-spectrum resistance refers to plant disease resistance against more than one pathogen species whereas “race-nonspecific” broad-spectrum resistance refers to plant disease resistance against multiple races or strains of the same pathogen species.

The terms “percent identity,” “% identity,” or “percent identical” as used herein in reference to two or more nucleotide or protein sequences is calculated by (i) comparing two optimally aligned sequences (nucleotide or protein) over a window of comparison, (ii) determining the number of positions at which the identical nucleic acid base (for nucleotide sequences) or amino acid residue (for proteins) occurs in both sequences to yield the number of matched positions, (iii) dividing the number of matched positions by the total number of positions in the window of comparison, and then (iv) multiplying this quotient by 100% to yield the percent identity. If the “percent identity” is being calculated in relation to a reference sequence without a particular comparison window being specified, then the percent identity is determined by dividing the number of matched positions over the region of alignment by the total length of the reference sequence. Accordingly, for purposes of the present application, when two sequences (query and subject) are optimally aligned (with allowance for gaps in their alignment), the “percent identity” for the query sequence is equal to the number of identical positions between the two sequences divided by the total number of positions in the query sequence over its length (or a comparison window), which is then multiplied by 100%. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity can be adjusted upwards to correct for the conservative nature of the substitution. Sequences that differ by such conservative substitutions are said to have “sequence similarity” or “similarity.” Sequences having a percent identity to a base sequence may exhibit the activity of the base sequence.

As used herein, the term “denoting” when used in reference to a plant genotype refers to any method whereby a plant is indicated to have a certain genotype. This includes any means of identification of a plant having a certain genotype. Indication of a certain genotype may include, but is not limited to, any entry into any type of written or electronic medium or database whereby the plant's genotype is provided. Indications of a certain genotype may also include, but are not limited to, any method where a plant is physically marked or tagged. Illustrative examples of physical marking or tags useful in the invention include, but are not limited to, a barcode, a radio-frequency identification (RFID), a label, or the like.

### VI. Deposit Information

A deposit of at least 625 seeds of Solanum pimpinellifolium line SPRJ2018 was made with the Provasoli-Guillard National Center for Marine Algae and Microbiota (NCMA), 60 Bigelow Drive, East Boothbay, Maine, 04544 USA. The deposit is assigned NCMA Accession No. 202302005, and the date of deposit was Feb. 3, 2023. Access to the deposit will be available during the pendency of the application to persons entitled thereto upon request. The deposit has been accepted under the Budapest Treaty and will be maintained in the NCMA Depository, which is a public depository, for a period of 30 years, or 5 years after the most recent request, or for the enforceable life of the patent, whichever is longer, and will be replaced if nonviable during that period. Applicant does not waive any infringement of their rights granted under this patent or any other form of variety protection, including the Plant Variety Protection Act (7 U.S.C. 2321 et seq.).

### VII. Further Embodiments

### Plants

E1: A Solanum lycopersicum plant comprising a recombinant chromosomal segment on chromosome 2 or a recombinant chromosomal segment on chromosome 5, wherein said chromosomal segment on chromosome 2 and said chromosomal segment on chromosome 5 each comprise a tomato spotted wilt virus (TSWV) resistance allele from Solanum pimpinellifolium, wherein said alleles confer increased resistance to resistance breaking isolates relative to a plant lacking said chromosomal segment.

E2: The plant of E1, comprising said chromosomal segment on chromosome 2.

E3: The plant of E1 or E2, comprising said chromosomal segment on chromosome 5.

E4: The plant of any one of E1 to E3, comprising said chromosomal segment on chromosome 2 and said chromosomal segment on chromosome 5.

E5: The plant of any one of E1 to E4, wherein said TSWV resistance allele on chromosome 2 is located within a chromosomal segment proximal to M4 (SEQ ID NO:4) in said plant.

E6: The plant of any one of E1 to E5, wherein said TSWV resistance allele on chromosome 2 is located between 1 bp and 31,168,278 bp of public tomato genome sequence SL5.0.

E7: The plant of any one of E1 to E6, wherein said TSWV resistance allele on chromosome 5 is located within a chromosomal segment distal to M11 (SEQ ID NO:11).

E8: The plant of any one of E1 to E6, comprising said chromosomal segment on chromosome 2 and said chromosomal segment on chromosome 5 and wherein said TSWV resistance allele on chromosome 2 is located between 1 bp and 31,168,278 bp of public tomato genome sequence SL5.0 and said TSWV resistance allele on chromosome 5 is located within a chromosomal segment distal to M11 (SEQ ID NO:11).

E9: The plant of any one of E1 to E8, wherein said TSWV resistance allele on chromosome 5 is located between 64,287,180 bp and 65,608,067 bp of public tomato genome sequence SL5.0.

E10: The plant of any one of E1 to E9, wherein: a) said recombinant chromosomal segment on chromosome 2 comprises a marker locus selected from the group consisting of marker locus M1 (SEQ ID NO:1), M2 (SEQ ID NO:2), M3 (SEQ ID NO:3), and M4 (SEQ ID NO:4); or b) said recombinant chromosomal segment on chromosome 5 comprises a marker locus selected from the group consisting of M8 (SEQ ID NO:8), M9 (SEQ ID NO:9), M10 (SEQ ID NO:10), and M11 (SEQ ID NO: 11).

E11: The plant of any one of E1 to E10, wherein said TSWV resistance alleles confer resistance to Sw-5b resistance breaking TSWV isolates.

E12: The plant of any one of E1 to E11, wherein the plant is homozygous for either or both of said TSWV resistance alleles.

E13: The plant of any one of E1 to E12, wherein the plant is heterozygous for either or both of said TSWV resistance alleles.

E14: The plant of any one of E1 to E13, wherein a sample of seed comprising said chromosomal segment on chromosome 2 and said chromosomal segment on chromosome 5 was deposited under NCMA Accession Number 202302005.

E15: A plant part of a plant of any one of E1 to E14, wherein the plant part is a cell, a seed, a root, a stem, a leaf, a head, a flower, a fruit, or pollen.

E15A: A plant as described in E15, wherein the plant part is a seed or a fruit of said plant.

E15B: A plant as described in E15, wherein the plant part is a seed or a fruit of a plant in accordance with E4, E8, or a combination of E8 with any one of E9 to E13.

### Methods

E16: A method for identifying a Solanum lycopersicum plant with resistance to tomato spotted wilt virus (TSWV), such as to resistance breaking tomato spotted wilt virus (TSWV) isolates, comprising: a) obtaining nucleic acids from at least a first Solanum lycopersicum plant; and b) identifying in said nucleic acids the presence of a recombinant chromosomal segment on chromosome 2 comprising a marker locus selected from the group consisting of marker locus M1 (SEQ ID NO:1), M2 (SEQ ID NO:2), M3 (SEQ ID NO:3), and M4 (SEQ ID NO:4); or a recombinant chromosomal segment on chromosome 5 comprising a marker locus selected from the group consisting of M8 (SEQ ID NO:8), M9 (SEQ ID NO:9), M10 (SEQ ID NO:10), and M11 (SEQ ID NO: 11); wherein each of said chromosomal segments comprises a tomato spotted wilt virus (TSWV) resistance allele from Solanum pimpinellifolium conferring resistance against TSWV.

E17: The method according to E16, comprising: a) obtaining nucleic acids from at least a first Solanum lycopersicum plant; and b) identifying in said nucleic acids the presence of a recombinant chromosomal segment on chromosome 2 comprises a marker locus selected from the group consisting of marker locus M1 (SEQ ID NO:1), M2 (SEQ ID NO:2), M3 (SEQ ID NO:3), and M4 (SEQ ID NO:4); and a recombinant chromosomal segment on chromosome 5 comprises a marker locus selected from the group consisting of M8 (SEQ ID NO:8), M9 (SEQ ID NO:9), M10 (SEQ ID NO:10), and M11 (SEQ ID NO: 11), wherein each of said chromosomal segments comprises a tomato spotted wilt virus (TSWV) resistance allele from Solanum pimpinellifolium conferring resistance against TSWV.

E18: The method according to E16 or E17, wherein step b) is carried out using marker locus M2 (SEQ ID NO:2).

E19: The method according to any one of E16 to E18, wherein step b) is carried out using marker locus M1 (SEQ ID NO:1).

E20: The method according to any one of E16 to E19, wherein step b) is carried out using marker locus M3 (SEQ ID NO:3).

E21: The method according to any one of E16 to E20, wherein step b) is carried out using marker locus M4 (SEQ ID NO:4).

E22: The method according to any one of E16 to E21, wherein step b) is carried out using marker locus M5 (SEQ ID NO:).

E23: The method according to any one of E16 to E22, wherein step b) is carried out using marker locus M6 (SEQ ID NO:6).

E24: The method according to any one of E16 to E23, wherein step b) is carried out using marker locus M7 (SEQ ID NO:7).

E25: The method according to any one of E16 to E24, wherein step b) is carried out using marker locus M8 (SEQ ID NO:8).

E26: The method according to any one of E16 to E25, wherein step b) is carried out using marker locus M9 (SEQ ID NO:9).

E27: The method according to any one of E16 to E26, wherein step b) is carried out using marker locus M10 (SEQ ID NO:10).

E28: The method according to any one of E16 to E27, wherein step b) is carried out using marker locus M11 (SEQ ID NO:11).

E29: The method according to any one of E16 to E28, wherein said recombinant chromosomal segment on chromosome 2 is located between 1 bp and 31,168,278 bp of public tomato genome sequence SL5.0.

E30: The method according to any one of E16 to E29, wherein said recombinant chromosomal segment on chromosome 5 is located between 64,287,180 bp and 65,608,067 bp of public tomato genome sequence SL5.0.

E31: The method according to any one of E16 to E30, wherein step b) comprises the identification of the homozygous (+/+) presence of the SPRJ2018 alleles on the recombinant chromosomal segment on chromosome 5.

E32: The method according to E31, wherein step b) comprises the identification of the homozygous (+/+) or heterozygous (+/−) presence of the resistance alleles from Solanum pimpinellifolium conferring resistance against TSWV, e.g., the SPRJ2018 alleles on the recombinant chromosomal segment on chromosome 2.

E33: The method according to E31 or E32, wherein step b) comprises the identification of the homozygous (+/+) presence of the resistance alleles from Solanum pimpinellifolium conferring resistance against TSWV, e.g., the SPRJ2018 alleles on the recombinant chromosomal segment on chromosome 2.

E34: The method according to any one of E16 to E33, wherein the resistance is determined by inoculating at least one plant to be identified, at least one susceptible control plant, and optionally at least one resistant control plant with TSWV inoculums using the following scale: Disease Index 1=hypersensitive reaction on the inoculated leaves only; Disease Index 3=clean plant, no symptoms; Disease Index 5=seedling with systemic chlorosis/necrosis, some mosaic/rings; Disease Index 7=stunted seedling with systemic chlorosis/necrosis, severe mosaic and top down wilt; Disease Index 9=Dead or dying seedling.

Examples for susceptible control plants are a variety that either does not contain any TSWV resistance genes, e.g. Celebrity and a variety containing the Sw-5b resistance gene such as the cultivar Stevens.

An example of a resistant control plant is SPRJ2018 (NCMA Accession No. 202302005).

The stage of the plants for such an assay to assess resistance is, e.g., a seedling of said (control) plants when the second true leaf is fully expanded. Inoculation with TSWV inoculums can be easily carried out, e.g., by rubbing said second true leaf with a finger or pestle dipped in TSWV inoculum. Plants for such an assay can be grown in soil or in a growth media, e.g. a 50/50 mix of Berger 2 and 6 growth media. The conditions should be kept constant such as 16 hours light and day/night temperature cycle of 25° C./18° C.

Plants can then be scored for disease symptoms, two weeks after inoculation using the above-described scale for Disease Index. The skilled person is well-aware how to determine the Disease Index of a plant. At the time of scoring, minimally 95% of the control plants should show the expected resistant or susceptible reaction.

Usually, an assay is carried out with more than one control plant per genotype, e.g., at least 10 plants, at least 15 plants such as 15 to 60 plants per genotype. When more than one plant of a genotype is used to evaluate the Disease Index, this value will be a mean value of the control plants with the same genotype.

E35: The method according to any one of E16 to E34, wherein the resistance is measured with an assay in accordance with an assay described in paragraphs [00141] to [00146] and the (mean) Disease Index of a Solanum lycopersicum plant with resistance to tomato spotted wilt virus to be identified is 5.2 or less.

E36: The method according to E35, wherein the Disease Index is 5 or less.

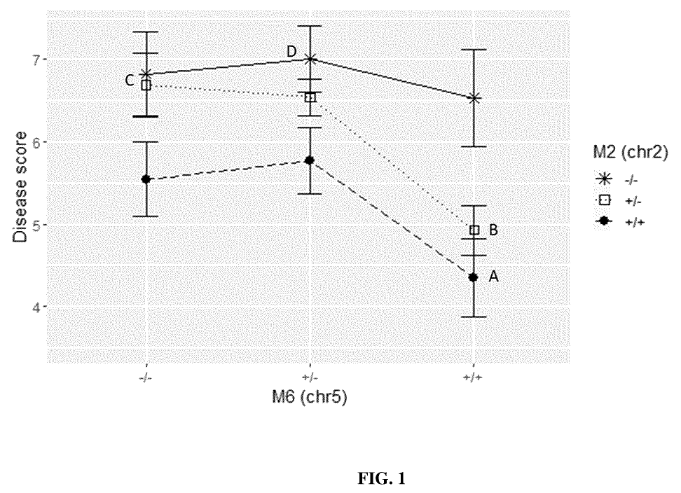

To identify the loci underlying this resistance, for example, bi-parental (rough) mapping can be conducted in an F₂ population derived from a cross between a resistant plant, e.g., SPRJ2018, and a susceptible processing tomato line (P1). The F₂ population, along with the parent lines, e.g., SPRJ2018 and P1, and their F₁ generation can be phenotyped for resistance and genotyped at markers across the genome. Interval (rough) mapping analysis identified regions on chromosome 2 and chromosome 5 that were significantly associated with disease resistance (see FIG. 1). To further validate the resistance provided by such QTLs, the resistance alleles from the resistant plant, e.g., SPRJ2018, can be introgressed into other susceptible elite tomato lines. In one embodiment, a resulting plant has a Disease Index value below 5. In another embodiment, a resulting plant has a Disease Index of below 4.5. Additional markers associated with the TSWV resistance loci disclosed herein (e.g. M1-M4 on chromosome 2 and M8-M11 on chromosome 5) are provided in Table 1. The column denoted as “Favorable Allele” shows the resistance allele for each marker.

Embodiments described for plants, methods for producing a plant, methods for selecting a plant in accordance with the present invention described herein can, if applicable, also be used for further defining the methods for identifying a plant in accordance with the present invention (see, e.g., further embodiments E16 to E35).

### EXAMPLES

The following disclosed embodiments are merely representative of the invention which may be embodied in various forms. Thus, specific structural, functional, and procedural details disclosed in the following examples are not to be interpreted as limiting.

### Example 1

### Pathology Assay for Testing for Resistance Breaking TSWV Resistance

TSWV resistance was measured using the assay described herein. TSWV inoculums were harvested from the new growth of infected plants that were 3-4 weeks old and showed visible TSWV symptoms. Inoculums were maintained on susceptible tomato or tobacco plants for short-term usage. For long-term storage 2 g of infected tissue was collected and stored at −80° C. To prepare the inoculums for use, 1 gram of infected fresh or defrosted tissue was ground in 5 ml of a standard phosphate buffer (0.1M K₂HPO₄, 0.1M KH₂PO₄, pH 7, and 0.01M Na₂SO₃) using a mortar and pestle to create a slurry, and ¼ tsp of celite was subsequently added.

15-60 plants per genotype, including susceptible and resistant controls were used. In particular, two susceptible controls are used to confirm the breaking nature of the isolate—a variety that does not contain any TSWV resistance genes, e.g. Celebrity, as well as a variety containing the Sw-5b resistance gene such as the cultivar Stevens. The resistant control used in this case was the SPRJ2018 source.

Plants were shown in a 50/50 mix of Berger 2 and 6 growth media and maintained in a growth room with 16 hours of light and day/night temperature cycle of 25° C./18° C. Seedlings were inoculated when the second true leaf is fully expanded by rubbing the leaf with a finger or pestle dipped in inoculum. Plants were rinsed with water 10 minutes after inoculation. The inoculum is viable for ˜30 minutes and should be kept on ice and stirred regularly. Plants were scored for disease symptoms 2 weeks after inoculation using the following scale: Disease Index 1=hypersensitive reaction on the inoculated leaves only; Disease Index 3=clean plant, no symptoms; Disease Index 5=seedling with systemic chlorosis/necrosis, some mosaic/rings; Disease Index 7=stunted seedling with systemic chlorosis/necrosis, severe mosaic and top down wilt; Disease Index 9=Dead or dying seedling. At the time of scoring, minimally 95% of the control plants should show the expected resistant or susceptible reaction.

### Example 2

### Mapping and Validation of QTLs from SPRJ2018 Conferring Resistance to Resistance Breaking Isolates of TSWV

The Solanum pimpinellifolium line designated SPRJ2018 was evaluated for TSWV resistance using the pathology assay described in Example 1 above. SPRJ2018 showed a high level of resistance to a resistance breaking TSWV isolate, with a mean disease score of 3 (symptomless) across several trials.

To identify the loci underlying this resistance, bi-parental rough mapping was conducted in an F₂ population derived from a cross between SPRJ2018 and a susceptible processing tomato line (P1). The F₂ population, along with the parent lines SPRJ2018 and P1, and their F₁ generation were phenotyped for resistance and genotyped at markers across the genome. Interval rough mapping analysis identified regions on chromosome 2 and chromosome 5 that were significantly associated with disease resistance (FIG. 1). The 1.5 LOD (i.e. logarithm of the odds) score support intervals spanning from 0 to 26.5 cM on chromosome 2 and from 80.5 to 121.8 cM on chromosome 5, these genetic positions refer to an internal genetic map. Using the public tomato reference genome SL5.0, the intervals are located from 0 to 34.80 Mb on chromosome 2, and from 64.1 to 65.6 Mb on chromosome 5. FIG. 1 shows the combined effect of the alleles with markers associated with the two novel QTLs from the rough mapping. Surprisingly, the presence of the resistance alleles at both identified loci increases the level of resistance, from a mean disease score of −7 (See point “C” on FIG. 1) to an improved mean score of −5 (See points “A” and “B” on FIG. 1). F₂ hybrids that were homozygous (+/+) for the SPRJ2018 alleles at both QTLs (“A” in FIG. 1) or homozygous for SPRJ2018 alleles at the QTL on chromosome 5 and heterozygous (+/−) on chromosome 2 (“B” in FIG. 1) were resistant with mean disease scores less than 5. F₂ hybrids homozygous for P1 alleles at both QTLs (“C” in FIG. 1) or heterozygous at one QTL and homozygous P1 at the other (“C” and “D” in FIG. 1) were susceptible (mean disease scores ˜7). Other two-locus genotypic combinations provide intermediate levels of resistance (FIG. 1).

As the initial QTL intervals were relatively large, a fine mapping approach was used to refine the regions comprising the resistance loci. The fine mapping utilized additional markers and provided additional information to refine the intervals and the markers linked with each QTL. Individuals comprising recombination events within one of the QTL intervals (as defined by the 1.5 LOD support intervals given above) were generated and fixed for the SPRJ2018 haplotype at the other QTL (e.g. recombinant on chromosome 2 and fixed SPRJ2018 on chromosome 5 and vice versa). The selected plants were selfed and progeny that were fixed for the recombination events were selected. These recombinants were tested in the pathology assay described in Example 1 above and scored for resistance to the resistance breaking TSWV isolate.

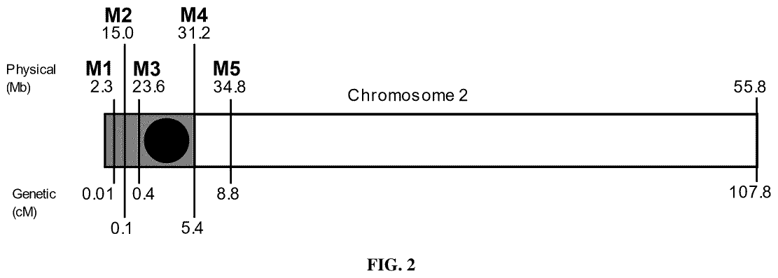

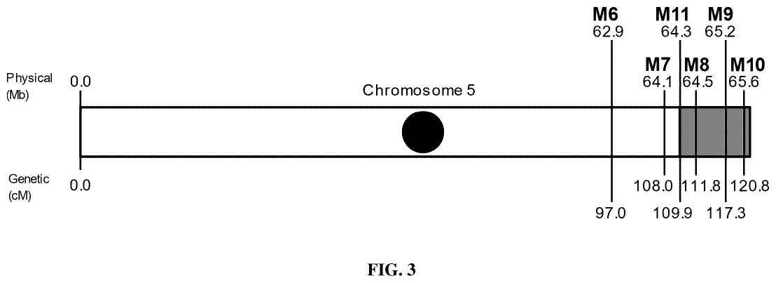

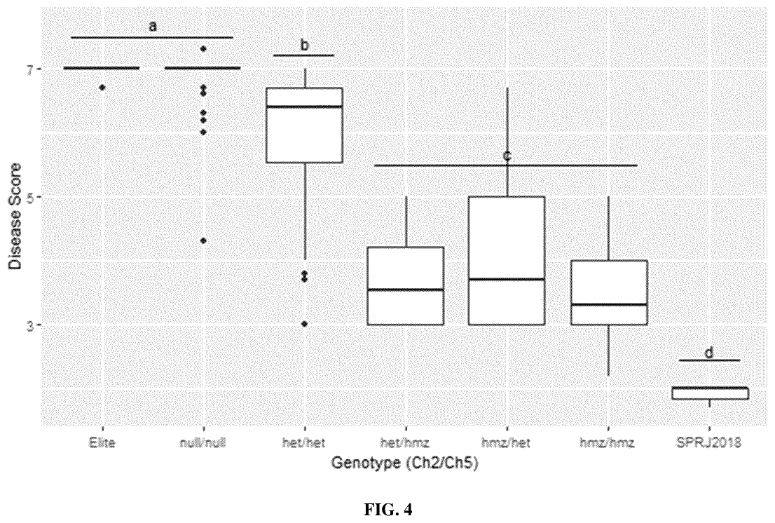

The minimal introgressions that provided resistance spanned from M1 to M4, with recombination breakpoints between M4 and M5 on chromosome 2; and from M11 or M8 to M10, with recombination breakpoints between M7 and M11/M8 on chromosome 5. These results refine both QTL intervals, the QTL on chromosome 2 (QTL2) is located between the start of chromosome 2 and marker M4, and the QTL on chromosome 5 (QTL5) is located between M11 and the end of chromosome 5, which respectively correspond to 0 to 5.41 cM (FIG. 2) and 109.9 cM to 120.8 cM (FIG. 3). Using the public tomato reference genome SL5.0, the novel QTLs described herein are located between the 0 to 31.2 Mb on chromosome 2; and 64.3 to 65.7 Mb on chromosome 5. To further validate the resistance provided by these intervals, the resistance alleles from SPRJ2018 were introgressed into three different susceptible elite tomato lines. Different genotypic combinations of these two QTLs were tested for resistance to the resistance breaking TSWV isolate. There were no significant differences between the three backgrounds nor were there significant differences between the plants that were either homozygous at both QTLs or homozygous at one of the QTLs and heterozygous at the other. Such plants showed high levels of resistance with mean disease scores between 3.5 and 4.0, demonstrating a remarkable difference when compared against susceptible lines which presented disease scores between 6.9 and 7.0 (FIG. 4). The identification and validation of these novel QTL allows for the development of new tomato varieties with improved resistance to the resistance breaking TSWV isolate. Additional markers associated with the TSWV resistance loci disclosed herein (e.g. M1-M4 on chromosome 2 and M8-M11 on chromosome 5) are provided in Table 1 below. The column denoted as “Fav. Allele” shows the resistance allele (favorable allele) from Solanum pimpinellifolium for each marker.

TABLE 1
List of markers and favorable alleles at each marker locus
for tracking TSWV resistance QTLs on chromosomes 2 and 5.

| | | Position of | | | SNP | | | |
|---|---|---|---|---|---|---|---|---|
| | | Marker | Public | | Position | | | Marker |
| | | Sequence on | Position of | Marker | in | | | Sequence |
| Marker | | SL5.0 | SNP | Size | Marker | SNP | Fav. | (SEQ ID |
| Name | Chr. | (bp) | (bp) | (bp) | (bp) | Change | Allele | NO) |
| M1 | 2 | 2,317,204- | 2,317,254 | 101 | 51 | [T/C] | C | 1 |
| | | 2,317,304 | | | | | | |
| M2 | 2 | 14,999,213- | 14,999,363 | 301 | 151 | [A/C] | C | 2 |
| | | 14,999,513 | | | | | | |
| M3 | 2 | 23,620,315- | 23,620,465 | 301 | 151 | [A/G] | G | 3 |
| | | 23,620,615 | | | | | | |
| M4 | 2 | 31,168,218- | 31,168,278 | 121 | 61 | [A/C] | C | 4 |
| | | 31,168,338 | | | | | | |
| M5 | 2 | 34,800,881- | 34,800,931 | 101 | 51 | [C/T] | T | 5 |
| | | 34,800,981 | | | | | | |
| M6 | 5 | 62,940,765- | 62,940,825 | 121 | 61 | [T/C] | C | 6 |
| | | 62,940,885 | | | | | | |
| M7 | 5 | 64,076,321- | 64,076,471 | 301 | 151 | [G/C] | C | 7 |
| | | 64,076,621 | | | | | | |
| M11 | 5 | 64,287,130- | 64,287,180 | 101 | 51 | [T/C] | C | 11 |
| | | 64,287,230 | | | | | | |
| M8 | 5 | 64,505,660- | 64,505,710 | 101 | 51 | [T/C] | C | 8 |
| | | 64,505,760 | | | | | | |
| M9 | 5 | 65,177,744 | 65,177,794 | 101 | 51 | [T/G] | G | 9 |
| | | 65,177,844 | | | | | | |
| M10 | 5 | 65,608,017- | 65,608,067 | 101 | 51 | [C/A] | A | 10 |
| | | 65,608,117 | | | | | | |

What is claimed is:

1. A Solanum lycopersicum plant comprising a recombinant chromosomal segment on chromosome 2 or a recombinant chromosomal segment on chromosome 5, wherein said chromosomal segment on chromosome 2 and said chromosomal segment on chromosome 5 each comprise a tomato spotted wilt virus (TSWV) resistance allele from Solanum pimpinellifolium, wherein said alleles confer increased resistance to resistance breaking isolates relative to a plant lacking said chromosomal segment.

2. The plant of claim 1, comprising said chromosomal segment on chromosome 2.

3. The plant of claim 1, comprising said chromosomal segment on chromosome 5.

4. The plant of claim 1, comprising said chromosomal segment on chromosome 2 and said chromosomal segment on chromosome 5.

5. The plant of claim 1, wherein said TSWV resistance allele on chromosome 2 is located within a chromosomal segment proximal to M4 (SEQ ID NO:4) in said plant.

6. The plant of claim 1, wherein said TSWV resistance allele on chromosome 2 is located between 1 bp and 31,168,278 bp of public tomato genome sequence SL5.0.

7. The plant of claim 1, wherein said TSWV resistance allele on chromosome 5 is located within a chromosomal segment distal to M11 (SEQ ID NO:11).

8. The plant of claim 1, wherein said TSWV resistance allele on chromosome 5 is located between 64,287,180 bp and 65,608,067 bp of public tomato genome sequence SL5.0.

9. The plant of claim 1, wherein:
a) said recombinant chromosomal segment on chromosome 2 comprises a marker locus selected from the group consisting of marker locus M1 (SEQ ID NO:1), M2 (SEQ ID NO:2), M3 (SEQ ID NO:3), and M4 (SEQ ID NO:4); or
b) said recombinant chromosomal segment on chromosome 5 comprises a marker locus selected from the group consisting of M8 (SEQ ID NO:8), M9 (SEQ ID NO:9), M10 (SEQ ID NO:10), and M11 (SEQ ID NO:11).

10. The plant of claim 1, wherein said TSWV resistance alleles confer resistance to Sw-5b resistance breaking TSWV isolates.

11. The plant of claim 1, wherein the plant is homozygous for either or both of said TSWV resistance alleles.

12. The plant of claim 1, wherein the plant is heterozygous for either or both of said TSWV resistance alleles.

13. The plant of claim 1, wherein a sample of seed comprising said chromosomal segment on chromosome 2 and said chromosomal segment on chromosome 5 was deposited under NCMA Accession Number 202302005.

14. A seed that produces the plant of claim 1.

15. A plant part of the plant of claim 1, wherein said plant part comprises either or both of said chromosomal segment on chromosome 2 and said chromosomal segment on chromosome 5.

16. The plant part of claim 15, wherein the plant part is a cell, a seed, a root, a stem, a leaf, a head, a flower, or pollen.

17. A method for producing a Solanum lycopersicum plant with resistance to resistance breaking tomato spotted wilt virus (TSWV) isolates, comprising introgressing into said plant a recombinant chromosomal segment on chromosome 2 or a recombinant chromosomal segment on chromosome 5, wherein said chromosomal segment on chromosome 2 and said chromosomal segment on chromosome 5 each comprise a TSWV resistance allele from Solanum pimpinellifolium, wherein said alleles confer increased resistance to resistance breaking isolates relative to a plant lacking said chromosomal segment.

18. The method of claim 17, wherein said introgressing comprises:
a) crossing a plant comprising said chromosomal segment on chromosome 2, said chromosomal segment on chromosome 5, or said chromosomal segment on chromosome 2 and said chromosomal segment on chromosome 5 with itself or with a second Solanum lycopersicum plant of a different genotype to produce one or more progeny plants; and
b) selecting a progeny plant comprising said chromosomal segment.

19. The method of claim 18, wherein selecting a progeny plant comprises detecting at least one allele proximal to M4 (SEQ ID NO:4) on chromosome 2; or distal to M11 (SEQ ID NO:11) on chromosome 5.

20. The method of claim 18, wherein selecting a progeny plant comprises detecting nucleic acids comprising:
a) marker locus M1 (SEQ ID NO:1), M2 (SEQ ID NO:2), M3 (SEQ ID NO:3), M4 (SEQ ID NO:4), or M5 (SEQ ID NO:5); or
b) marker locus M6 (SEQ ID NO:6), M7 (SEQ ID NO:7), M8 (SEQ ID NO:8), M9 (SEQ ID NO:9), M10 (SEQ ID NO:10), or M11 (SEQ ID NO:11).

21. The method of claim 17, wherein said resistance comprises resistance to Sw-5b resistance breaking TSWV isolates.

22. The method of claim 18, wherein the progeny plant is an F₂-F₆ progeny plant.

23. The method of claim 18, wherein said crossing comprises backcrossing, marker-assisted selection, or assaying for said TSWV resistance.

24. The method of claim 23, wherein said backcrossing comprises from 2-7 generations of backcrosses.

25. The method of claim 17, wherein a sample of seed comprising said chromosomal segment on chromosome 2 and said chromosomal segment on chromosome 5 was deposited under NCMA Accession Number 202302005.

26. A Solanum lycopersicum plant obtainable by the method of claim 17.

27. A method of selecting a Solanum lycopersicum plant exhibiting resistance to resistance breaking tomato spotted wilt virus (TSWV) isolates, comprising:
a) screening one or more plants with at least one nucleic acid marker to detect a polymorphism genetically linked to said TSWV resistance; and
b) selecting one or more plants comprising said polymorphism genetically linked to said TSWV resistance, wherein said polymorphism is within or genetically linked to a chromosomal segment proximal to M4 (SEQ ID NO:4) in said plant on chromosome 2; or a chromosomal segment distal to M11 (SEQ ID NO:11) in said plant on chromosome 5.

28. The method of claim 27, wherein selecting one or more plants comprises detecting at least one polymorphism:
a) at a locus selected from the group consisting of marker locus M1 (SEQ ID NO:1), M2 (SEQ ID NO:2), M3 (SEQ ID NO:3), M4 (SEQ ID NO:4), and M5 (SEQ ID NO:5); or
b) at a locus selected from the group consisting of marker locus M6 (SEQ ID NO:6), M7 (SEQ ID NO:7), M8 (SEQ ID NO:8), M9 (SEQ ID NO:9), M10 (SEQ ID NO:10), and M11 (SEQ ID NO:11).

29. The method of claim 28, wherein screening one or more plants comprises PCR, single strand conformational polymorphism analysis, denaturing gradient gel electrophoresis, cleavage fragment length polymorphism analysis, TAQMAN assay, and/or DNA sequencing.

30. The method of claim 29, wherein the Solanum lycopersicum plant comprises said chromosomal segment on chromosome 2 or said chromosomal segment on chromosome 5, and wherein a sample of seed comprising said chromosomal segment on chromosome 2 and said chromosomal segment on chromosome 5 was deposited under NCMA Accession Number 202302005.
